(19) United States
(12) Patent Application Publication
(10) Pub. No.: US 2020/0315980 A1
(43) Pub. Date: Oct. 8, 2020

(54) Drug Delivery System Comprising Nanocarrier Loaded with Urate Oxidase and Metal-Based Nanoparticle Capable of Degrading Hydrogen Peroxide and Pharmaceutical Composition Comprising the Same

(71) Applicant: Gwangju Institute of Science and Technology, Buk-gu (KR)

(72) Inventors: Giyoong Tae, Buk-gu (KR); Inchan Kwon, Buk-gu (KR); Manse Kim, Buk-gu (KR); Seoungkyun Kim, Buk-gu (KR); Kiyoon Kwon, Buk-gu (KR); Secheon Jung, Buk-gu (KR)

(21) Appl. No.: 16/839,204

(22) Filed: Apr. 3, 2020

(30) Foreign Application Priority Data
Apr. 8, 2019 (KR) 10-2019-0040944

(51) Int. Cl. A61K 9/51; C12N 9/06

(52) U.S. Cl. CPC A61K 9/5115; A61K 9/5146; A61K 38/00; C12Y 107/03003; A61K 9/5192; C12N 9/0012

(57) ABSTRACT

The present disclosure is directed to a drug delivery system in which urate oxidase and metal-based nanoparticles for hydrogen peroxide degradation are loaded in a temperature-sensitive nanocarrier, and a pharmaceutical composition for treating hyperuricemia-related disease comprising the drug delivery system. In the drug delivery system of the present disclosure, urate oxidase and metal-based nanoparticles for hydrogen peroxide degradation are positioned close to each other, thereby effectively removing the toxic substance hydrogen peroxide (H₂O₂) generated during uric acid degradation. The drug delivery system of the present disclosure may be developed as an active ingredient of a drug for preventing or treating hyperuricemia-related disease.

### CROSS-REFERENCE TO RELATED APPLICATION

This application claims priority to Korean Patent Application No. 10-2019-0040944, filed on Apr. 8, 2019, the disclosure of which is incorporated herein by reference in its entirety.

### BACKGROUND

### Technical Field

The present disclosure relates to a drug delivery system in which urate oxidase and metal-based nanoparticles for hydrogen peroxide degradation are loaded in a nanocarrier, a pharmaceutical composition for treating hyperuricemia comprising the drug delivery system, and a method for producing the drug delivery system.

### Description of the Related Art

Therapeutic proteins are used for clinical treatment of various human diseases due to their favorable properties such as biocompatibility, selectivity and efficacy compared to chemical drugs. However, therapeutic proteins can cause side effects such as headache, diarrhea, temporary rash, reduced blood cells, cardiotoxicity, hypertension, hypersensitivity, exfoliative dermatitis, immunogenicity and serum disease. Reducing the side effects of drugs is an important issue in developing therapeutic proteins, along with other existing issues such as improving therapeutic efficacy and stability. To date, alteration of amino acid composition or conjugation of biocompatible molecules (e.g., polyethylene glycol (PEG)) has been extensively applied to reduce the dose of therapeutic protein and thus reduce side effects. However, this approach can compromise the critical properties of therapeutic proteins, and hence alternative strategies need to be developed.

Urate oxidase (UOX) is an enzyme that catalyzes the reaction of converting insoluble uric acid (UA) into soluble 5-hydroxyisoacetate (5-HIU). UOX is used to treat acute or chronic gout, gouty redness, gouty arthritis, kidney disease, cardiovascular disease, and hyperuricemia associated with tumor lysis syndrome (TLS). Tumor lysis syndrome (TLS) can occur during cancer treatment when many tumor cells are lysed. Gout is a common inflammatory arthritis caused by the deposition of uric acid crystals in joints and soft tissues. About 0.1 to 10% of the population worldwide suffers from gout. The conversion of UA to 5-HIU, catalyzed by UOX, also generates H₂O₂, which is toxic and can adversely affect patients deficient in glucose-6-phosphate dehydrogenase.

Gold (Au) nanoparticles (AuNPs) are highly biocompatible metal nanoparticles and are known to degrade hydrogen peroxide (H₂O₂), like catalase. Such enzyme-mimicking nanoparticles are called nanozymes. Nanozymes are attracting attention as an alternative to enzymes because of their high stability and cost effectiveness compared to enzymes. However, the enzyme-nanozyme system has been mainly used in the biosensor field because of its limitations in in vivo applications. For example, the use of a catalase-mimicking AuNP with UOX reduced H₂O₂ levels in vitro, but the application thereof in vivo is difficult. When UOX and AuNP are administered to blood, they are diluted to very low concentrations. Hence, they cannot be located close enough to each other to achieve effective cascade reactions consisting of 1) the generation of H₂O₂ by UOX and 2) the removal of H₂O₂ by AuNP. In order to encounter H₂O₂, a by-product to be efficiently removed by AuNP, AuNP and UOX need to be loaded together into a single carrier.

The patent documents and references cited herein are incorporated herein by reference to the same extent as if each reference were individually and specifically indicated to be incorporated by reference.

### PRIOR ART DOCUMENTS

### Patent Documents

(Patent Document 1) WO 2009/120707

### Non-Patent Documents

(Non-Patent Document 1) W. He, Y. Zhou, W. G. Wamer, X. Hu, X. Wu, Z. Zheng, M. D. Boudreau, J. Yin, Intrinsic catalytic activity of Au nanoparticles with respect to hydrogen peroxide decomposition and superoxide scavenging, Biomaterials 2012, 34(3), 765-773.

### SUMMARY

The present inventors have made extensive efforts to develop a drug delivery system capable of effectively treating hyperuricemia in blood. As a result, the present inventors have experimentally demonstrated that when urate oxidase and catalytic metal nanoparticles capable of degrading hydrogen peroxide are loaded together in a nanocarrier having temperature-sensitive properties and administered in vivo, blood uric acid levels can be effectively lowered, thereby completing the present disclosure.

Therefore, an object of the present disclosure is to provide a drug delivery system capable of effectively treating hyperuricemia.

Another object of the present disclosure is to provide a pharmaceutical composition for preventing or treating hyperuricemia or hyperuricemia-related disease.

Still another object of the present disclosure is to provide a method for producing the drug delivery system.

Other objects and technical features of the present disclosure will be more clearly understood from the following detailed description of the disclosure, the appended claims and the accompanying drawings.

In accordance with one aspect of the present disclosure, there is provided a drug delivery system comprising: (i) urate oxidase; (ii) metal-based nanoparticles for hydrogen peroxide degradation; and (iii) a temperature-sensitive nanocarrier loaded with (i) and (ii).

In the present disclosure, “urate oxidase” is an enzyme that catalyzes the conversion of uric acid to a more soluble product, that is, 5-hydroxyisoacetate (5-HIU) which is more easily secreted out of the body.

As used herein, the term “metal-based nanoparticles for hydrogen peroxide degradation” refers to a metal-based catalytic capable of promoting a reaction in which hydrogen peroxide (H₂O₂) is degraded into water (H₂O) and oxygen (O₂).

According to an embodiment of the present disclosure, the metal-based nanoparticles for hydrogen peroxide degradation may comprise metal, metal ion with chelate or metal oxide. The metal may comprise for example, gold (Au), platinum (Pt), manganese (Mn), silver (Ag), or iron (Fe), but is not limited thereto. Preferably, the metal for hydrogen peroxide degradation may be gold (Au), platinum (Pt), or silver (Ag). The metal ion with chelate comprise for example, manganese ion (Mn2+), Prussian Blue (PB) or iron ion (Fe2+). The metal oxide comprise for example, manganese oxide (MnO2), iron oxide (Fe2O3) or ceria (CeO2).

According to an embodiment of the present disclosure, the metal for hydrogen peroxide degradation may comprise, for example, gold (Au), platinum (Pt), manganese (Mn), silver (Ag), or iron (Fe), but is not limited thereto. Preferably, the metal for hydrogen peroxide degradation may be gold (Au), platinum (Pt), or silver (Ag).

As used herein, the term “temperature-sensitive” refers to a property in which the size of the nanocarrier decreases with increasing temperature and the size of the nanocarrier increases with decreasing temperature.

In the present disclosure, the diameter of the temperature-sensitive nanocarrier increases with decreasing temperature, whereas the diameter of the temperature-sensitive nanocarrier decreases with increasing temperatures.

In the present disclosure, this increase or decrease in the diameter of the nanocarrier with increasing or decreasing temperature is reversible.

In the present disclosure, the size of pores formed in the nanocarrier changes as the diameter of the nanocarrier increases or decreases. For example, when a drug to be delivered is encapsulated into the nanocarrier with increased pore size at a low temperature (e.g., 4° C.), and then applied to the human body, the pore size decreases. Due to such temperature-sensitive properties, when the drug delivery system of the present disclosure is administered to the human body, the distance between the urate oxidase and the metal-based nanoparticles for hydrogen peroxide degradation, loaded into the nanocarrier, may be decreased and the urate oxidase and the metal-based nanoparticles for hydrogen peroxide degradation may be positioned closer to each other. This proximity between the urate oxidase and the metal-based nanoparticles allows the rapid and efficient degradation of hydrogen peroxide.

According to one embodiment of the present disclosure, the temperature-sensitive nanocarrier is a Pluronic-based nanocarrier.

In the present disclosure, the “Pluronic” comprises a triblock copolymer composed of poly(ethylene oxide)-poly(propylene oxide)-poly(ethylene oxide) (PEO-PPO-PEO).

According to another embodiment of the present disclosure, the triblock copolymer composed of PEO-PPO-PEO may further comprise a photo-crosslinkable functional group.

According to a specific embodiment of the present disclosure, the Pluronic may be diacrylated Pluronic F127.

In one embodiment of the present disclosure, the weight ratio between the urate oxidase and the metal-based nanoparticles for hydrogen peroxide degradation, loaded in the temperature-sensitive nanocarrier, is 1 (urate oxidase): 0.15 to 1.5 (metal-based nanoparticles for hydrogen peroxide degradation).

In accordance with another aspect of the present disclosure, there is provided a pharmaceutical composition for preventing or treating hyperuricemia or hyperuricemia-related disease comprising: (i) a therapeutically effective amount of a drug delivery system in which urate oxidase and metal-based nanoparticles for hydrogen peroxide degradation are loaded in a temperature-sensitive nanocarrier; and (ii) a pharmaceutically acceptable carrier.

In the present disclosure, the term “therapeutically effective amount” means an amount sufficient and suitable to treat hyperuricemia or hyperuricemia-related disease in vivo by administering the drug delivery system to a patient.

The pharmaceutical composition of the present disclosure may comprise a pharmaceutically acceptable carrier in addition to the active ingredient “drug delivery system”. Such carriers are those that are commonly used in formulation, and include, but are not limited to, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, menthol and mineral oil.

The pharmaceutical composition of the present disclosure may further comprise a lubricant, a wetting agent, a sweetener, a flavoring agent, an emulsifying agent, a suspending agent, a preservative, etc., in addition to the above-described components. Suitable pharmaceutically acceptable carriers and formulations are described in detail in Remington's Pharmaceutical Sciences (19th ed., 1995).

The suitable dose of the pharmaceutical composition of the present disclosure may vary depending on factors such as formulation method, mode of administration, and patient's age, weight, sex, disease conditions, diet, duration of administration, route of administration, rate of excretion, and response sensitivity.

The pharmaceutical composition of the present disclosure may be administered orally or parenterally. For parenteral administration, the pharmaceutical composition may be administered by intravenous injection, subcutaneous injection, intramuscular injection, intraperitoneal injection, transdermal administration, or the like.

Meanwhile, considering that the pharmaceutical composition of the present disclosure is applied for the treatment or prevention of hyperuricemia or hyperuricemia-related disease, the pharmaceutical composition of the present disclosure may be administered by various oral or parenteral routes.

The concentration of the drug delivery system, which is an active ingredient contained in the pharmaceutical composition of the present disclosure, may be determined in consideration of the purpose of treatment, the condition of a patient, the period of time required, etc., and is not limited to a specific range of concentration.

The pharmaceutical composition of the present disclosure may be prepared in a unit dosage form by formulation using a pharmaceutically acceptable carrier and/or excipient according to a method which can be easily carried out by a person having ordinary skill in the technical field to which the present disclosure pertains, or may be prepared by filling into a multi-dose container. The formulations may be in the form of solutions, suspensions or emulsions in oils or aqueous media, or in the form of extracts, powders, granules, tablets or capsules, and may additionally comprise dispersing or stabilizing agents.

In the present disclosure, the hyperuricemia-related disease may be selected from the group consisting of acute or chronic gout, gout redness, gouty arthritis, kidney disease, cardiovascular disease, and tumor lysis syndrome (TLS).

In still another aspect of the present disclosure, there is provided a method for producing a drug delivery system comprising steps of:

(a) preparing urate oxidase and metal-based nanoparticles for hydrogen peroxide degradation; and

(b) loading a temperature-sensitive nanocarrier with the prepared urate oxidase and metal-based nanoparticles for hydrogen peroxide degradation.

According to one embodiment of the present disclosure, step (b) in the method for producing the drug delivery system may be performed at a low temperature so that the size of the temperature-sensitive nanocarrier is increased.

According to a specific embodiment of the present disclosure, the low temperature at which the size of the temperature-sensitive nanocarrier is increased may be 1 to 10° C., preferably 2 to 8° C., more preferably 3 to 5° C., and even more preferably 4° C.

### BRIEF DESCRIPTION OF THE DRAWINGS

FIG. 1A is a schematic view showing that when uric acid (UA) is converted into 5-HIU by uric acid oxidase (UOX), toxic substance H₂O₂ is generated and can cause cell damage in tissues of hyperuricemia patients.

FIG. 1B shows cascade reactions between UOX and gold nanoparticles (AuNP).

FIG. 1C shows that when UOX and AuNP are loaded and encapsulated in a nanocarrier (NC) and co-administered, efficient degradation of UA may occur in vivo due to effective removal of H₂O₂.

FIG. 2A is a graph showing the degradation rate of UA by UOX in the presence of various concentrations of AuNP encapsulated in a nanocarrier (NC).

FIG. 2B is a graph comparing the size of a nanocarrier (NC) with that of UOX-AuNP@NC in the case in which UOX and AuNP were loaded in the nanocarrier (NC) to form a drug delivery system (UOX-AuNP@NC).

FIG. 2C shows a graph showing the change in surface charge of a nanocarrier (NC) and UOX-AuNP@NC after loading UOX and AuNP in the nanocarrier (NC), and shows the change in PDI value after loading UOX and AuNP.

FIG. 3A is a graph showing the results of analyzing UA degradation by comparing the cascade reactions of UOX and AuNP in different systems.

FIG. 3B is a graph showing the results of analyzing relative H₂O₂ generation by comparing the cascade reactions of UOX and AuNP in different systems.

FIG. 4A is a graph showing the cytotoxicity of UOX-AuNP@NC in the absence of UA.

FIG. 4B is a graph showing the results of comparing cytotoxicity in the presence of UA in various systems, including a UOX-AuNP@NC system, to evaluate cytotoxicity induced by H₂O₂ (# p>0.05, *p<0.05, **p<0.01, t-test and ANOVA).

FIG. 5A is a schematic view showing an experimental process for comparing the hyperuricemia treatment effects of various enzyme systems, including a UOX-AuNP@NC system, in vivo using a mouse model.

FIG. 5B is a graph comparing the hyperuricemia treatment effects of various enzyme systems, including a UOX-AuNP@NC system, in vivo.

FIG. 6 is a graph showing that the particle size of UOX-AuNP@NC is maintained at a substantially constant level in 10% serum-containing medium (37° C., 100 rpm) up to 7 days.

FIG. 7 is a graph showing residual UOX activity in blood 18 hours after administration of five enzyme systems.

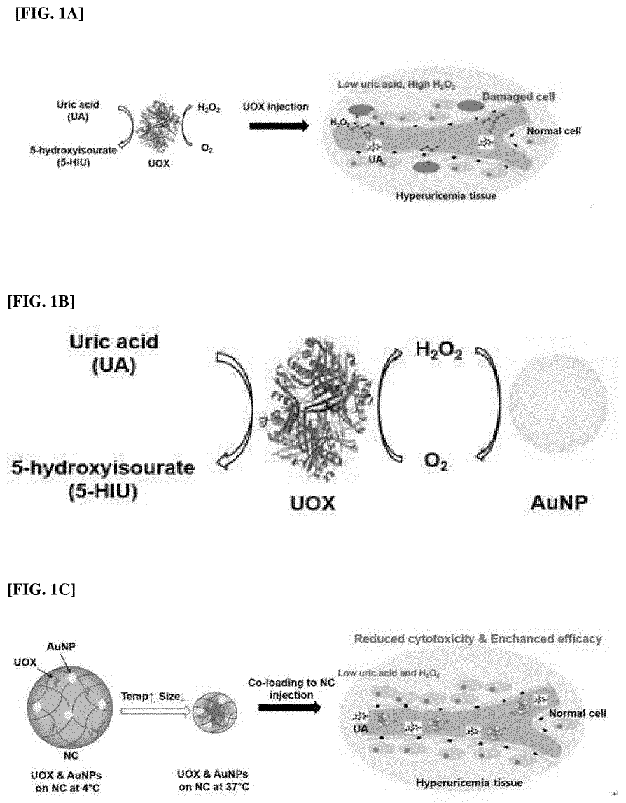

### DESCRIPTION OF SPECIFIC EMBODIMENTS

Specific embodiments described herein are intended to represent preferred embodiments or examples of the present disclosure, and the scope of the present disclosure is not limited thereto. It is obvious to those skilled in the art that modifications and other uses of the present disclosure do not depart from the scope of the present disclosure as defined in the appended claims.

### EXAMPLES

Experimental Materials and Method

1. Materials

Gold nanoparticles (diameter: 5 nm) coated with poly(vinylpyrrolidone) were purchased from nanoComposix Inc. (San Diego, Calif.). Ni-nitrilotriacetic acid (Ni-NTA) resin was purchased from Qiagen (Valencia, Calif., USA). A Vivaspin centrifugal concentrator with a molecular weight cutoff (MWCO) of 50 kDa was purchased from Sartorius Corporation (Bohemia, N.Y., USA). A PD-10 desalting column was purchased from GE Health Care (Piscataway, N.J., USA). Yeast extract, tryptone and agar were purchased from DB Biosciences (San Jose, Calif., USA). Pluronic F127 of poly(ethylene oxide)-poly(propylene oxide)-poly(ethylene oxide) (PEO-PPO-PEO) (PEO100-PPO65-PEO100, molecular weight: 12.6 kDa) was obtained from BASF Corporation (Seoul, Korea). Acryloyl chloride was purchased from Tokyo Chemical Industry (TCI, Tokyo, Japan). 4-(2-hydroxyethoxy)phenyl-(2-hydroxy-2-propyl) ketone (Irgacure 2959) was obtained from Ciba Specialty Chemicals Inc. (Basel, Switzerland). A Nanosep centrifugal device (MWCO 300 kDa) for spin filtration was purchased from Pall Life Sciences (Ann Arbor, Mich., USA). Unless otherwise indicated, all other reagents were purchased from Sigma-Aldrich (Saint Louis, Mo., USA). All reagents were of analytical grade and were used without further purification.

2. Purification of Urate Oxidase (UOX) and Production of Gold Nanoparticles (AuNP)

UOX was prepared as follows. Briefly, the plasmid-encoding UOX gene (pQE80-UOX) was transformed into TOP10 Escherichia coli cells. The transformed TOP10_pQE80-UOX cells were cultured for UOX expression. Cells expressing UOX were harvested and subjected to immobilized metal ion affinity chromatography using a hexahistidine affinity tag attached to UOX. The purity of UOX is equal to or more than 95%, as analyzed by SDS-PAGE analysis. The catalytic activity of the purified UOX was analyzed by uric acid degradation assay.

More specifically, the plasmid encoding a recombinant urate oxidase (UOX) gene derived from Aspergillus flavus under the control of T5 promoter (pQE80-UOX) was transformed into TOP10 E. coli cells. The pre-cultured transformed cells were inoculated into a fresh 2×YT medium containing 100 μg/mL of ampicillin, and shake-cultured at 220 rpm and at 37° C. When the optical density at 600 nm (OD_{600 nm}) reached 0.5, 1 mM isopropyl β-D-1-thiogalactopyranoside was added to the culture for induction of UOX expression and incubated for 5 hours. Then, the cells were pelleted by centrifugation at 6,000 rpm for 10 minutes. The cell pellets were stored at −80° C. until use, resuspended in lysis buffer (50 mM sodium phosphate, 0.3 M sodium chloride, 10 mM imidazole, pH 7.4), and incubated with 100 μg/mL of lysozyme on ice for 10 minutes. The resuspended cell pellets were sonicated on ice for 15 min (10-sec pulse on and 20-sec pulse off). The cell lysate was centrifuged at 12,000 rpm at 4° C. for 40 minutes, and then Ni-NTA resin was added to the supernatant which was then incubated at 4° C. for 30 minutes. The supernatant incubated with the Ni-NTA resin was loaded onto a polypropylene column and washed with washing buffer (50 mM sodium phosphate, 0.3 M NaCl, 20 mM imidazole, pH 7.4). After washing, UOX was eluted with elution buffer (50 mM sodium phosphate, 0.3 M sodium chloride, 250 mM imidazole, pH 7.4). The eluted UOX solution was buffer-exchanged with PBS buffer (pH 7.4) using a PD-10 column. The UOX concentration was determined by absorbance measurement at 280 nm using Synergy™ multimode microplate reader (BioTek, Winooski, Vt., USA) according to Beer-Lambert's law. The extinction coefficient of UOX was 53,520 M⁻¹cm⁻¹.

Before encapsulation into nanocarriers (NC), the catalase-mimic catalytic activities of AuNPs were measured by H₂O₂ degradation assay.

3. Determination of Degradation Rate of Uric Acid by UOX (Urate Oxidase)

In order to optimize the ratio of UOX to AuNPs, various concentrations of AuNPs (1, 2.5, 5, 10, 15, 20 and 25 μg/mL) were mixed with 500 nM UOX. To determine the degradation rate of UA, 200 μM UA was mixed with UOX in PBS buffer (pH 7.4), and the absorbance at 293 nm was monitored using Synergy™ multi-mode microplate reader. The extinction coefficient of UA at 293 nm was 12,300 M⁻¹cm⁻¹.

4. Production of UOX-AuNP@NCs

Pluronic-based nanocarriers (NCs) were synthesized by photo-crosslinking the micelle state of diacrylated pluronic F127 (DA-F127).

Briefly, a 10 wt % DA-F127 solution was prepared in deionized water (DIW). Then, 0.154 mL of the DA-F127 solution was added to 1.846 mL of DIW containing 0.057 wt % of irgacure 2959 to induce micelle formation of DA-F127. Using an ultraviolet (UV) lamp (VL-4.LC, 8 W, Vilber Lourmat, France), a photocrosslinking process for NC synthesis was performed by irradiation at 1.3 mW/cm² for 15 minutes. The produced NCs were purified by dialysis for 1 day and freeze-dried. UOX and AuNPs were co-encapsulated into the nanocarriers (NCs) using the temperature-dependent size change of the NCs (UOX-AuNP@NCs).

Briefly, 1 mg of NCs was dissolved in 1 mL of a 5 μM UOX solution containing 50 μg of AuNPs. The mixture was incubated overnight at 4° C. to induce the size expansion of the NCs. Then, the temperature of the mixed solution was increased to 37° C. and maintained for 10 minutes to reduce the size of the NCs for encapsulation. The final solution was centrifuged using a Nanosep centrifugal device to remove unloaded UOX and AuNPs (11,000 rpm, 37° C., 10 min). Then, the amount of UOX in UOX-AuNP@NCs was measured by bicinchoninic acid protein assay (Micro BCA protein analysis kit, Thermo Fisher Scientific, Waltham, Mass., USA). The loaded amount of AuNPs in UOX-AuNP@NCs was calculated by absorbance of AuNPs at 525 nm using a spectrometer (Molecular Devices, Spectra-MaxM2e, Trenton, N.J., USA).

5. Size and Surface Charge of UOX-AuNP@NC

The sizes and surface charges of NCs and UOX-AuNP@NCs were analyzed at 37° C. using an electrophoretic light scattering system (ELS-8000, Otsuka Electronics Osaka, Japan). Then, the stability of UOX-AuNP@NCs was determined by measuring the size change of UOX-AuNP@NCs at 1 mg/mL in a cell culture medium (RPMI 1640, Gibco, N.Y., USA) containing 10% fetal bovine serum (FBS) at 37° C. and 100 rpm. The size of UOX-AuNP@NCs was observed at predetermined time points for 7 days.

6. Determination of In Vitro UA Degradation Rate in NCs

In order to compare the UA degradation efficiencies, four enzyme systems were prepared as follows: (1) UOX, (2) UOX in NCs (UOX@NC), (3) UOX with AuNPs (UOX-AuNP), and (4) UOX-AuNP in NCs (UOX-AuNP@NC). Enzyme samples were incubated at 37° C. for 15 minutes and enzymatic assays of UOX at 10 nM were performed. 200 μL of assay buffer (50 mM sodium borate buffer, pH 8.0, 100 μM UA) was used and the UA degradation rates were calculated by measuring absorbance change at 293 nm.

7. Determination of H₂O₂ Levels In Vitro

Concentrations of H₂O₂ generated by UOX systems (UOX, UOX@NC, UOX-AuNP, or UOX-AuNP@NC) when adding UA to the samples were estimated using degradation of N,N-dimethyl-4-nitroaniline (RNO). 0.25 M of RNO and 0.03 M of histidine were prepared in DIW and mixed together. Then, UOX, UOX@NC, UOX-AuNP or UOX-AuNP@NC solution was added to the RNO solution. The concentrations of UOX and UA were fixed at 200 nM and 1 mM, respectively. Then, H₂O² generation from each sample was measured by incubating the sample at room temperature for 10 minutes and observing N,N-dimethyl-4-nitroaniline (RNO) concentration by measuring absorbance at 440 nm.

8. In Vitro Cytotoxicity of UOX-AuNP@NC

Cytotoxicity of UOX-AuNP@NCs without UA was examined using squamous cell carcinoma-7 (SCC7) cells obtained from American Type Culture Collection (Rockville, Md., USA). First, SCC7 cells were seeded at 5×10³ cells/well in a 96-well tissue culture plate with a cell culture medium (RPMI 1640, Gibco, N.Y., USA) containing 10% FBS and 1% penicillin-streptomycin. The seeded cells were incubated for 24 hours at 37° C. under a 5% CO₂ condition. Then, the cells were washed with PBS buffer (pH 7.4) and treated with the prepared UOX-AuNP@ NC samples at different concentrations (from 0 to 1 mg/mL on the basis of NC concentration). The sample-treated cells were incubated overnight at 37° C. Then, the cells were washed with PBS buffer, and the cytotoxicity of the UOX-AuNP@NCs without UA was calculated using a Cell Counting Kit-8 (CCK-8, Dojindo Molecular Technologies, Inc., Kumamoto, Japan).

9. In Vitro Cytotoxicity Determination of H₂O₂ Generated by the UOX Systems

To examine the cytotoxicity of H₂O₂ generated by the UOX systems, the cytotoxicity of UOX-AuNP@NC in the presence of UA was compared to that of UOX, UOX@NC and UOX-AuNP. SCC7 cells were seeded at 5×10³ cells/well in a 96-well tissue culture plate and cultured overnight at 37° C. under 5% CO₂. The cultured cells were washed with PBS and treated with 100 μL of each of samples such as UOX, UOX@NC, UOX-AuNP and UOX-AuNP@NC. Each sample was processed to a final concentration of 200 nM UOX and 1 mM UA. Among them, UOX@NC was a negative control. The sample-treated cells were incubated for 3 hours at 37° C. and washed with PBS. The cytotoxicity of sample groups in the presence of UA was determined using a Cell Counting Kit-8.

10. Determination of In Vivo Uric Acid Level

For in vivo experiments, 8-week old C57BL/6 mice (Orient Bio Inc., Seongnam, South Korea) were used. To induce a hyperuricemia state in the mice, the mice were treated with hypoxanthine and potassium oxonate. Before the induction of hyperuricemia, all the mice were fasted overnight. Hypoxanthine was dissolved in 3% soluble starch solution and injected intragastrically into the mice at a dose of 475 μg/g. Then, potassium oxonate was dissolved in a co-solvent composed of a mixture of lanolin and liquid paraffin (3:2, v/v) and injected subcutaneously into the hypoxanthine-treated mice at a dose of 95 μg/g.

Five groups of mice were prepared: (1) normal mice (control), (2) untreated hyperuricemia mice, (3) hyperuricemia mice treated with UOX, (4) hyperuricemia mice treated with UOX-AuNP, and (5) hyperuricemia mice treated with UOX-AuNP@NC. 100 μL of sample solution (UOX concentration was fixed at 1 μM) of either PBS, UOX, UOX-AuNP or UOX-AuNP@NC was injected into the tail vein of the hyperuricemia mice. At predetermined time points (pre-induction and 1, 3, 6, 12, and 18 hours after injection), blood was collected from the sample-treated mice via retro-orbital bleeding. At 18 hours after injection of samples, blood was collected by cardiac puncture. The collected blood was centrifuged at 4° C. and 3000 rpm for 5 minutes to obtain serum. The UA concentration in the serum was analyzed after sample injection to compare the effect of UA degradation by sample treatment. For measuring the serum level of uric acid, samples were analyzed by the previously reported method with little modification. Serum samples were mixed with 0.3 M acetate buffer (pH 3.6) and then stored at 4° C. until analysis. Reagent mixtures for determination of serum UA were prepared in 0.3 M acetate buffer (pH 3.6) at a concentration of 0.83 mM 2,4,6-tripyridyl-s-triazine (TPTZ) and 1.66 mM FeCl₃.H₂O. The absorbance at 593 nm was measured to determine the UA concentration. In addition, in the presence or absence of NCs, the UA degradation rates of serum samples at 18 hours after sample injection were measured to evaluate the residual activity of UOX.

Experimental Results

1. Physiochemical Properties and Optimal Ratio of UOX and AuNPs Encapsulated in NCs

In the present disclosure, Pluronic-based NCs were used to deliver UOX and AuNPs simultaneously. UOX and AuNPs were co-encapsulated in NCs through temperature-dependent size changes. In order to achieve efficient UA degradation by UOX in the presence of AuNPs, the optimal ratio of UOX to AuNPs was examined. Various concentrations of AuNPs (1, 2.5, 5, 10, 15, 20, and 25 μg/mL) were mixed with 500 nM UOX, and then encapsulated in NCs. In all cases, the loading efficiencies were 90% or higher.

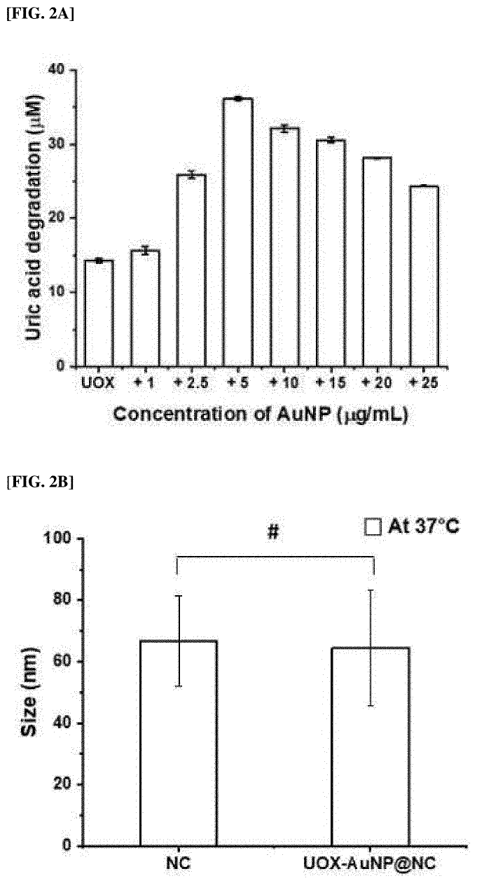

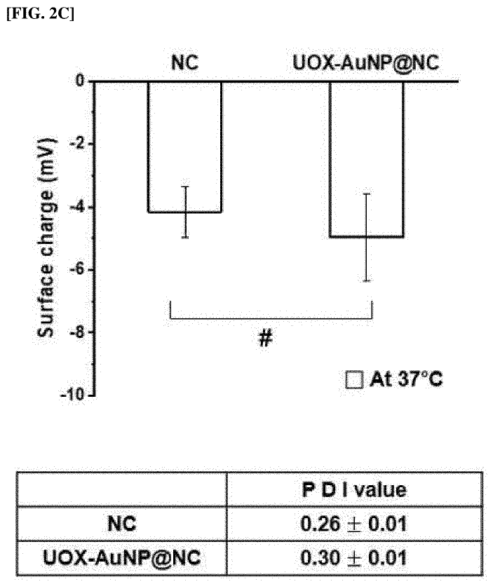

The enzymatic activity assay of samples showed that 5 μg/mL was the optimal concentration of AuNPs for efficient UA degradation (see FIG. 2A). In the presence of 5 μg/mL AuNPs, the amount of UA degraded by UOX was 2.5 times higher than that by UOX alone. The simple and efficient loading of various components into Pluronic-based NCs made it possible to modulate the composition of active components in the carrier and easily determine the optimal ratio between UOX and AuNPs. The ratio of UOX to AuNPs was fixed at the optimal ratio in all subsequent experiments. Under these conditions, the loading efficiency of UOX and AuNPs into the NCs was 90% or higher (92±8% AuNPs and 93±1% UOX, respectively).

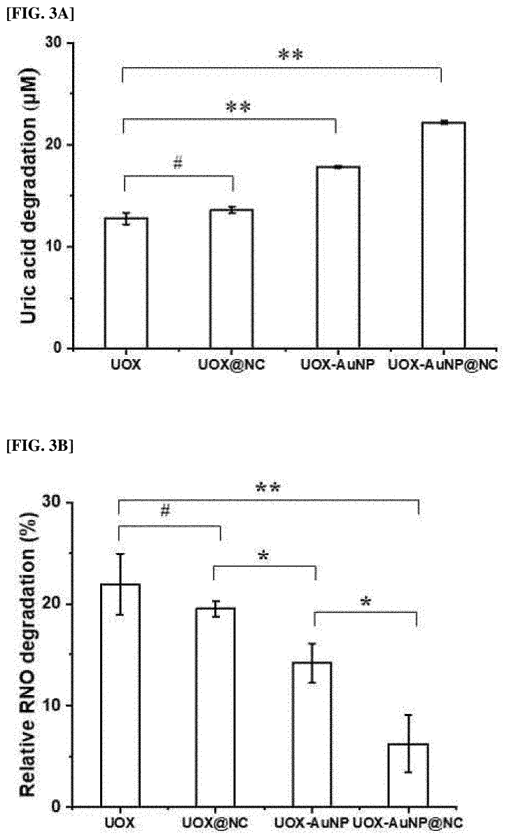

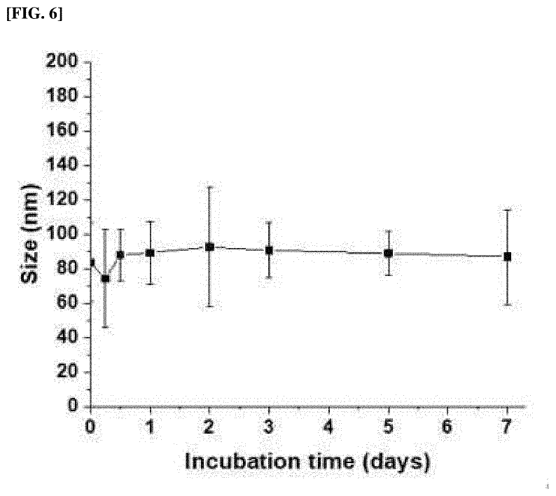

The size and surface charge of the produced NCs were analyzed using an electrophoretic light scattering system at 37° C. to predict the size of NCs in the body. When UOX and AuNPs were not loaded, the size and surface charge of NCs were 67±15 nm and −4.2±0.8 mV, respectively (see FIGS. 2B and 2C). After encapsulating UOX and AuNPs in NC (UOX-AuNP@NC), the size and surface charge of UOX-AuNP@NCs were 65±19 nm and −5.1±1.7 mV, respectively (see FIGS. 2B and 2C), indicating that there was no significant change in the size and surface charge of NC upon loading of UOX and AuNPs. Efficient loading of AuNPs into NCs, instead of adsorption to the surface of NCs, was confirmed. In addition, considering the predicted net charge of UOX as +1.4 at pH 7.4, no increase but similar surface charge of NCs after loading both UOX and AuNPs supports that UOX together with AuNPs was loaded inside the NCs, instead of adsorption onto NC surfaces. In addition, UOX-AuNP@NC showed good size stability in a serum-containing medium (10% serum, 37° C., 100 rpm) up to 7 days (FIG. 6). This implies colloidal stability in vivo. Considering that Pluronic-based NCs can preserve protein activity and capture gold nanomaterials without perturbation of properties such as absorbance shift, it was speculated that the physicochemical properties of UOX and AuNPs are not substantially affected by the encapsulation of UOX and AuNPs. In FIG. 3A, the catalytic activity of UOX in NCs was not substantially reduced compared with that of free UOX. Similarly, the AuNPs in NCs exhibited catalytic activity comparable to that of free AuNPs. Therefore, it is expected that NCs simultaneously deliver UOX and AuNPs without compromising the critical properties in an in vivo environment.

2. Enhanced Catalytic Activities of UOX Encapsulated Together with AuNPs in NCs

The present inventors analyzed the change in catalytic efficiency of UOX upon co-encapsulation with AuNPs in NCs. It was hypothesized that the efficiency of catalytic activity will be enhanced by two factors. One is that the addition of AuNPs will degrade the side-product H₂O₂, accelerating the UA degradation reaction. The other is the proximity effect of multiple catalysts in cascade reactions. Co-encapsulation of UOX and AuNPs in NCs will greatly reduce the distance between UOX and AuNPs, accelerating H₂O₂ degradation in situ. In order to prove these hypotheses, four groups of enzyme systems were prepared, including UOX alone (UOX), UOX encapsulated in NC (UOX@NC), a free mixture of UOX and AuNPs (UOX-AuNP), and UOX and AuNPs encapsulated in NCs (UOX-AuNP@NC). The degraded amounts of UA and H₂O₂ were measured. As shown in FIG. 3A, UOX and UOX@NC showed similar UA degradation rates. In the presence of AuNPs, UA degradation by UOX was 40% higher than that by UOX alone. In addition, UOX-AuNP@NC degraded 70% more UA than UOX alone. It was observed that UA degradation rate by UOX in NCs was similar to that by UOX alone.

Next, the present inventors examined whether different enzyme systems led to different H₂O₂ levels during UA degradation in vitro. H₂O₂ levels were estimated by measuring RNO degradation (FIG. 3B). In the case of UOX alone, almost 20% of RNO was degraded via H₂O₂ produced from UA degradation by UOX. Under the same reaction conditions, RNO degradation by UOX@NC was not significantly different from that by UOX alone. However, in the presence of AuNPs (UOX-AuNP), approximately 14% of RNO was degraded, indicating the lower H₂O₂ level. This result was consistent with the hypothesis of in situ degradation of H₂O₂ by AuNPs. Interestingly, when both UOX and AuNP were loaded into NCs (UOX-AuNP@NC), only about 5% of RNO was degraded, implying more efficient removal of H₂O₂ than UOX-AuNP. These RNO degradation assay results (FIG. 3B) were well consistent with UA degradation assay results (FIG. 3A).

Similar UA degradation and H₂O₂ production by UOX alone and UOX@NC indicate that NCs themselves did not substantially affect the enzymatic activity of UOX. More importantly, these results imply that NCs do not hinder the transport of UA as well as H₂O₂ across NCs or inside NCs. Thus, NCs not only provided the efficient loading of enzyme and nanoparticles, but also allowed the transport of small molecules including substrate and product for enzymatic reaction across or inside the carrier, which is a key requirement for proper action of delivered enzymes against target molecules present inside the body. A significant increase in the UA degradation and a significant decrease in H₂O₂ level of UOX-AuNP@NC compared to UOX-AuNP imply that NCs did not hinder the cascade reactions between them. In addition, these results demonstrate the hypothesis of the proximity effect made by co-localizing UOX and AuNPs in NCs on H₂O₂ removal and UA degradation. The present inventors speculated that the distances between UOX and AuNPs were closer in NCs than those between a free mixture of UOX and AuNPs. Considering that the blood volume is much greater than the sample injection volume, samples are expected to be substantially diluted in vivo upon administration. Therefore, the more pronounced effect of co-encapsulation of UOX and AuNPs in NCs on H₂O₂ removal and UA degradation would be expected in vivo. Through the above-described experimental results, the present inventors confirmed the favorable features of the Pluronic-based delivery platform having enhanced efficacy.

3. In Vitro Cytotoxicity of UOX-AuNP@NC with or without UA

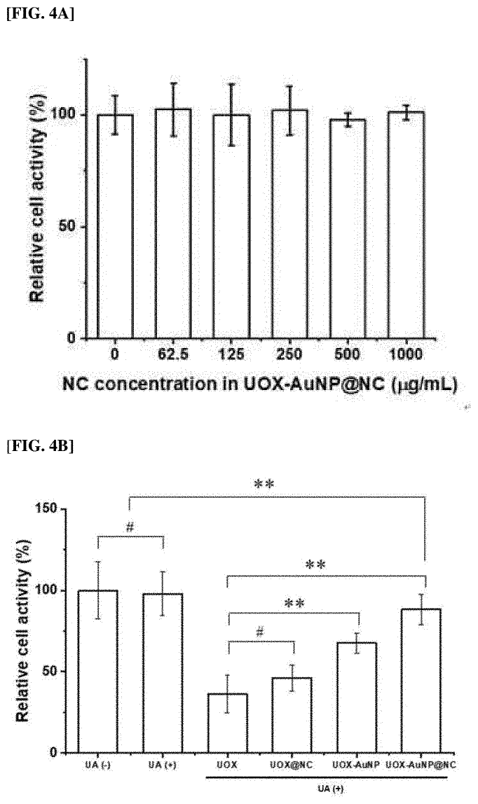

Before the in vivo experiment using UOX-AuNP @NC, the cytotoxicity of UOX-AuNP@NC without UA was measured to verify the biosafety of the nanosystem using SCC7 cancer cells (FIG. 4A). The cytotoxicity was indicated by reduction in the cellular activity. The assay results showed that UOX-AuNP@NC without UA exhibited no cytotoxicity up to 1 mg/mL based on the NC concentration. Considering that NCs themselves have no in vitro toxicity, AuNP does not have severe toxicity issue, and UOX can produce toxic substance such as H₂O₂ only in the presence of UA. Thus, UOX-AuNP@NC, containing both UOX and AuNP inside the Pluronic-based NC, is expected to be safe without significant toxicity in the absence of UA.

In order to evaluate H₂O₂-related cytotoxicity, the cytotoxicity of UOX-AuNP @ NC system was compared to those of other samples in the presence of UA (FIG. 4B). SCC7 cancer cells were used because cancer cells are more susceptible to reactive oxygen species such as H₂O₂ accumulation than normal cells. As expected, UA alone samples (UA (+)) did not show any significant cytotoxicity, similar to negative control samples without UA (UA (−)). In contrast, in the presence of UA, UOX samples showed significant (about 70%) cell death due to the produced H₂O₂. Encapsulation of UOX in NCs (UOX@NC) did not significantly reduce the cytotoxicity, similar to the results of H₂O₂ production (FIG. 3B). On the other hand, UOX and AuNPs dissolved solution (UOX-AuNP) samples showed the significantly reduced cytotoxicity (about 40%) due to H₂O₂ removal by AuNPs in the closed experimental setup. More importantly, UOX-AuNP@NC showed significantly reduced cytotoxicity compared to UOX-AuNP. The cellular activity of UOX-AuNP@NC samples was very high, and was even comparable to that of the control UA (−). All of these results were well consistent with the results of UA degradation assay (FIG. 3A) and H₂O₂ production assay (FIG. 3B). In addition, these results suggest that H₂O₂ generated by UOX during UA degradation can cause toxicity, a potential side-effect in in vivo clinical applications; however, efficient removal of H₂O₂ by UOX-AuNP@NC can significantly mitigate the toxicity issue of H₂O₂ produced by UOX.

4. Evaluation of In Vivo Activities of NC Loaded with UOX and AuNP

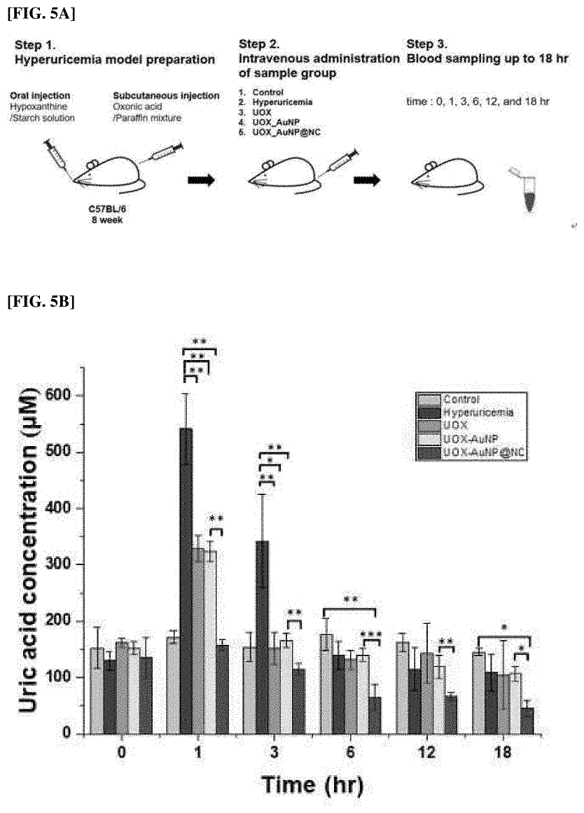

To evaluate the therapeutic effect of UOX-AuNP @NC in vivo, a hyperuricemia mouse model was used, as shown in FIG. 5A. Mice were divided into the following groups: normal control (Control), hyperuricemia (Hyperuricemia), UOX-treated (UOX), UOX-AuNP-treated (UOX-AuNP), and UOX-AuNP@NC-treated (UOX-AuNP@NC) groups. C57BL/6 hyperuricemia mice were injected with UOX, UOX-AuNP, or UOX-AuNP@ NC by intravenous administration with the same amount of enzyme. Blood samples obtained at predetermined time points were analyzed and the concentrations of UA were determined using a calibration curve (FIG. 5B).

First, among the experimental groups, the hyperuricemia group showed a much higher (over 3-fold) UA concentration in serum at 1 hour compared to the pre-induction (0 hour) or the normal control (no hyperuricemia) group (FIG. 5B). At 3 hours after induction of hyperuricemia, a 2-fold or more increase in UA concentration was observed for the hyperuricemia group compared to pre-induction (0 hour) or the normal control group. At 6 hour after induction of hyperuricemia, the hyperuricemia group showed similar UA concentrations in serum compared to that of the control group or the pre-induction state. Thus, the method for hyperuricemia induction induced an increase in the UA concentration initially, then lasted at least up to 3 hours. This temporal effect on hyperuricemia was also reported in previous reports using the similar induction method. In contrast, UOX-treated groups (UOX, UOX-AuNP, and UOX-AuNP@NC) all showed significantly lower concentrations of UA than that of the hyperuricemia group at both 1 hour and 3 hours, confirming the UA degradation by UOX in vivo. Among UOX-treated groups, UOX and UOX-AuNP showed very similar UA concentrations (similar UA degradation), whereas UOX-AuNP@NC showed a significantly lower concentration of UA than UOX and UOX-AuNP, revealing a superior UA degradation by UOX-AuNP@NC compared to UOX and UOX-AuNP (FIG. 5B). The similar effects of UOX and UOX-AuNP suggest that simple mixing and co-injection of UOX and AuNP was not sufficient to achieve the cascade reaction of UOX and AuNP in vivo. Thus, as expected, dilution after injection seemed to prevent the co-localization of UOX and AuNP in vivo. In contrast, much more effective degradation of UA by UOX-AuNP@ NC compared to UOX and UOX-AuNP proved that co-delivery of UOX and AuNP by NC encapsulation could make a sufficient environment for the cascade reaction to occur in blood.

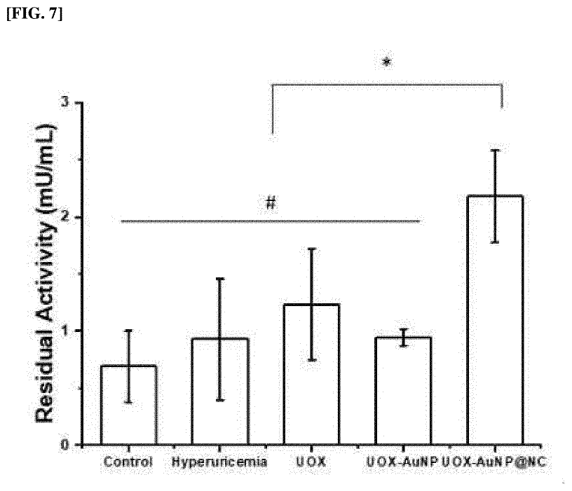

In addition, at 6 hours or later, the UOX and UOX-AuNP groups showed similar UA concentrations compared to the normal or hyperuricemia group in contrast to the maintenance of lower concentration of UA for the UOXAuNP@NC group, indicating that only the UOX-AuNP@NC group was effective up to 18 hours. Considering the biodistribution results of Pluronic-based NCs, which showed that when administered intravenously, the administered NC stayed in the main organ (predominantly in the liver) for at least 1 day in the previous experiment, the residual UA degradation effect at a later time point appeared to be because the in vivo half-life of UOX and AuNP was extended by encapsulation into NCs. At 18 hours, the residual UOX activity in serum by UOX-AuNP@NC was confirmed (FIG. 7).

Clearance of the toxic intermediate H₂O₂ can facilitate UA degradation. Also, the effective removal of H₂O₂ by UOX-AuNP@NC can lower the toxicity issue associated with H₂O₂. Thus, the UA degradation effect was significantly enhanced by co-delivering UOX and AuNP using NC (UOX-AuNP@NC) in vivo, and the delivery platform of the present disclosure has an obvious clinical potential for hyperuricemia treatment through synergistic effect of UOX and AuNPs.

The effects and advantages of the present disclosure are summarized as follows.

(i) The present disclosure is directed to a drug delivery system in which urate oxidase and metal-based nanoparticles for hydrogen peroxide degradation are loaded in a temperature-sensitive nanocarrier, and a pharmaceutical composition for treating hyperuricemia-related disease comprising the drug delivery system.

(ii) In the drug delivery system of the present disclosure, urate oxidase and metal-based nanoparticles for hydrogen peroxide degradation are positioned close to each other, thereby effectively removing the toxic substance hydrogen peroxide (H₂O₂) generated during uric acid degradation.

(iii) The drug delivery system of the present disclosure may be developed as an active ingredient of a drug for preventing or treating hyperuricemia-related disease.

Although the present disclosure has been described in detail with reference to specific features, it will be apparent to those skilled in the art that this description is only of a preferred embodiment thereof, and does not limit the scope of the present disclosure. Thus, the substantial scope of the present disclosure will be defined by the appended claims and equivalents thereto.

What is claimed is:

1. A drug delivery system comprising: (i) urate oxidase; (ii) metal-based nanoparticles for hydrogen peroxide degradation; and (iii) a temperature-sensitive nanocarrier loaded with (i) and (ii).

2. The drug delivery system of claim 1, wherein the metal based is metal, metal ion with chelate or metal oxide.

3. The drug delivery system of claim 2, wherein the metal is gold (Au), platinum (Pt), manganese (Mn), silver (Ag) or iron (Fe).

4. The drug delivery system of claim 2, wherein the metal ion with chelate is manganese ion (Mn2+), Prussian Blue (PB) or iron ion (Fe2+).

5. The drug delivery system of claim 2, wherein the metal oxide is manganese oxide (MnO2), iron oxide (Fe2O3) or ceria (CeO2).

6. The drug delivery system of claim 1, wherein the temperature-sensitive is a property in which the size of the nanocarrier decreases with increasing temperature and the size of the nanocarrier increases with decreasing temperature.

7. The drug delivery system of claim 6, wherein the temperature-sensitive nanocarrier is a Pluronic-based nanocarrier.

8. The drug delivery system of claim 7, wherein the Pluronic is a triblock copolymer comprising poly(ethylene oxide)-poly(propylene oxide)-poly(ethylene oxide) (PEO-PPO-PEO).

9. The drug delivery system of claim 8, wherein the triblock copolymer comprising PEO-PPO-PEO further comprises a photo-crosslinkable functional group.

10. The drug delivery system of claim 1, wherein the weight ratio between the urate oxidase and the metal-based nanoparticles for hydrogen peroxide degradation is 1 (urate oxidase): 0.15 to 1.5 (metal-based nanoparticles for hydrogen peroxide degradation).

11. A pharmaceutical composition for preventing or treating hyperuricemia or hyperuricemia-related disease comprising: (i) a therapeutically effective amount of the drug delivery system of claim 1; and (ii) a pharmaceutically acceptable carrier.

12. The pharmaceutical composition of claim 11, wherein the hyperuricemia-related disease is a disease selected from the group consisting of acute or chronic gout, gouty redness, gouty arthritis, kidney disease, cardiovascular disease, and tumor lysis syndrome (TLS).

13. A method for producing a drug delivery system comprising steps of:
(a) preparing urate oxidase and metal-based nanoparticles for hydrogen peroxide degradation; and
(b) loading a temperature-sensitive nanocarrier with the prepared urate oxidase and metal-based nanoparticles for hydrogen peroxide degradation.

14. The method of claim 13, wherein step (b) is performed at a low temperature so that the size of the temperature-sensitive nanocarrier is increased.
